(19) United States
(12) Patent Application Publication
(10) Pub. No.: US 2020/0078487 A1
(43) Pub. Date: Mar. 12, 2020

(54) ODOR CONTROL DEVICE

(71) Applicant: Eric Blumenthal, Cleveland, OH (US)

(72) Inventor: Eric Blumenthal, Cleveland, OH (US)

(21) Appl. No.: 16/561,101

(22) Filed: Sep. 5, 2019

Related U.S. Application Data
(60) Provisional application No. 62/728,257, filed on Sep. 7, 2018.

(51) Int. Cl. A61L 9/12

(52) U.S. Cl. CPC A61L 9/12; A61L 2209/133

(57) ABSTRACT

An odor control device is provided. The odor control device has a holder unit. The holder unit has a holder body. The holder body has a holder body first surface and an oppositely facing holder body second surface. The holder body second surface has at least one holder locking element. A replaceable cartridge is infused with a deodorizer. The cartridge has at least one cartridge locking element for engaging the holder locking element. The cartridge locking element is at least one of integrally formed with and integrally formed in the cartridge. The at least one holder locking element is selectively engaged with the at least one cartridge locking element to at least partially maintain the cartridge in the holder unit.

### RELATED APPLICATION

This application claims priority from U.S. Provisional Application No. 62/728,257, filed 7 Sep. 2018, the subject matter of which is incorporated herein by reference in its entirety.

### TECHNICAL FIELD

This disclosure relates to an odor control device.

### BACKGROUND

A problem common to both homeowners and industry is masking or removing offensive odors also within a closed space. Conventional methods of making or removing the offensive odors include the use of aerosols and slow release products. These slow release products are generally placed, attached to, or hung in the closed space.

### SUMMARY

In an aspect, an odor control device is provided. The odor control device has a holder unit. The holder unit has a holder body. The holder body has a holder body first surface and an oppositely facing holder body second surface. The holder body second surface has at least one holder locking element. A replaceable cartridge is infused with a deodorizer. The cartridge has at least one cartridge locking element for engaging the holder locking element. The cartridge locking element is at least one of integrally formed with and integrally formed in the cartridge. The at least one holder locking element is selectively engaged with the at least one cartridge locking element to at least partially maintain the cartridge in the holder unit.

In an aspect, an odor control device is provided. A holder unit has a holder body. The holder body has a holder body first surface and an oppositely facing holder body second surface. The holder body second surface has at least two holder locking elements. A replaceable cartridge is infused with a deodorizer and at least partially formed from a deformable and elastomeric material. The cartridge has at least two cartridge locking elements. The cartridge is selectively deflectable from a biased condition to selectively engage at least a portion of each of the at least two cartridge locking elements to at least a portion of a corresponding one of the at least two holder locking elements. The engagement of the at least two cartridge locking elements to the at least two holder locking elements at least partially maintains the cartridge in the holder unit.

In an aspect, an odor control device is provided. A holder unit has a holder body. The holder body has a holder body first surface and an oppositely facing holder body second surface. The holder body second surface has at least one holder locking element. A replaceable cartridge is infused with a deodorizer. The cartridge has at least one cartridge locking element for receiving the holder locking element. When the at least one holder locking element is selectively received in the at least one cartridge locking element, the cartridge is selectively laterally rotatable in a first direction to at least partially engage the at least one cartridge locking element with the at least one holder locking element. The engagement of the at least one cartridge locking element with the at least one holder locking element at least partially maintains the cartridge in the holder unit.

### BRIEF DESCRIPTION OF THE DRAWINGS

For a better understanding, reference may be made to the accompanying drawings, in which:

FIG. 1 is a side view of an element of an odor control device according to one aspect of the present invention;

FIG. 2 is a perspective top view of the element of the aspect of FIG. 1;

FIG. 3 is a perspective bottom view of the element of the aspect of FIG. 1;

FIG. 4 is a perspective top view of an element of the aspect of FIG. 1, in a first configuration;

FIGS. 5-9 illustrate an example sequence of operation of a portion of the aspect of FIG. 4;

FIG. 10 is a perspective bottom view of an element of the aspect of FIG. 1, in a second configuration;

FIG. 11 is a bottom view of the element of the aspect of FIG. 10;

FIG. 12 is a cross-sectional view taken along line 12-12 of FIG. 11; and

FIGS. 13-15 illustrate an example sequence of operation of a portion of the aspect of FIG. 10.

### DESCRIPTION OF EMBODIMENTS

Unless defined otherwise, all technical and scientific terms used herein have the same meaning as is commonly understood by one of ordinary skill in the art to which the present disclosure pertains.

As used herein, the term “user” can be used interchangeably to refer to an individual who prepares for, assists with, and/or performs the operation of a tool.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, “contacting,” etc., another element, it can be directly on, attached to or contacting the other element or intervening elements may also be present. It will also be appreciated by those of skill in the art that references to a structure or feature that is disposed “adjacent” another feature may not have portions that overlap or underlie the adjacent feature.

Spatially relative terms, such as “over” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “over” other elements or features would then be oriented “under” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

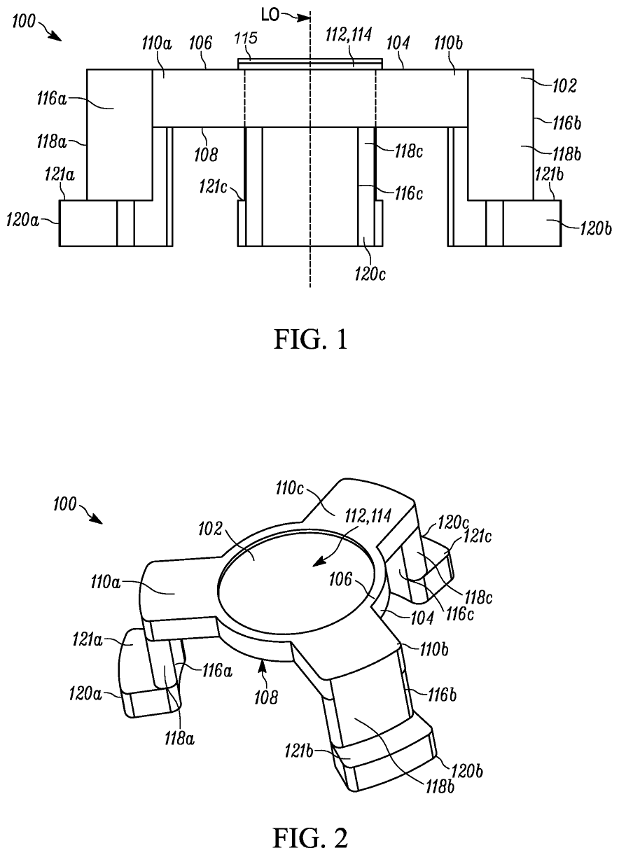

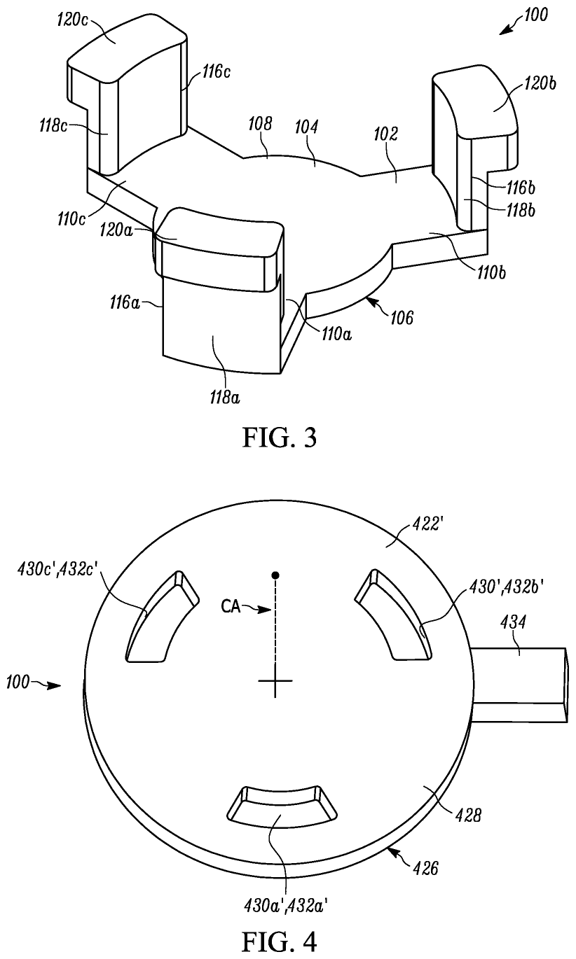

An odor control device 100 is provided. As shown in FIGS. 1-3, the odor control device 100 includes a holder unit 102 having a holder body 104. The holder body 104 has longitudinally spaced and oppositely facing holder body first and second surfaces 106, 108. The term “longitudinal” is used herein to indicate a substantially vertical direction, in the orientation of FIG. 1. The holder body 104 may have at least one holder body branch 110 that laterally extends from the holder body 104. The term “lateral” is used herein to indicate a direction substantially perpendicular to the “longitudinal” direction, and is shown as the horizontal direction in the orientation of FIG. 1. As shown in FIGS. 1-3, the holder body 104 may have first, second, and third holder body branches 110a, 110b, 110c that may be laterally spaced about a holder body longitudinal axis LO so that the holder unit 102 is rotationally symmetrical.

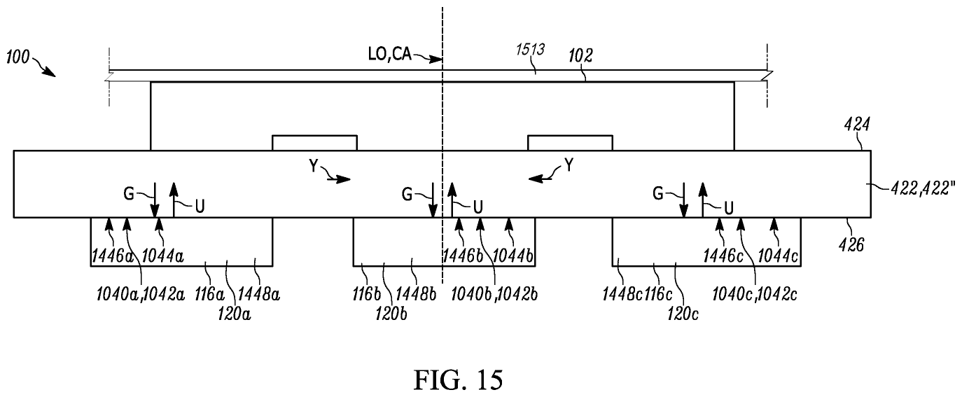

The holder body first surface 106 may have at least one attachment element 112 for selectively attaching the holder unit 102 to a support surface 1513 (FIG. 15), such as, but not limited to, a lid and/or a sidewall of a trash receptacle. The holder unit 102 may be selectively removably attached to the support surface 1513 such that a predetermined amount of an applied force may be necessary to selectively remove the holder unit 102 from the support surface 1513. In such case, the holder unit 102 remains attached to the support structure until the predetermined amount of the applied force is applied to the holder unit 102 to selectively remove the holder unit 102 from the support structure. Instead of being selectively removably attached to the support structure through the attachment element 112, the holder unit 102 may be substantially permanently attached to the support structure through the attachment element 112. Alternatively, the holder unit 102 may not include an attachment element 112, and instead, the holder unit 102 may be provided as an integral part of the support surface 1513. In such case, the support surface 1513 may comprise at least a portion of the holder body first surface 106. For example, the support surface 1513 could be an inner lid or other portion of a trash receptacle, and at least a portion of the holder body first surface 106 could be provided as a feature during manufacture of the trash receptacle.

The at least one attachment element 112 may be an adhesive 114. The adhesive 114 may be covered by a selectively removable cover 115 when the odor control device 100 is in a stored condition. Although the attachment member 112 has been described as including the adhesive 114, the attachment member 112 may also or instead include at least one of a clip, an adhesive, a hook and loop attachment member, a magnetic attachment member, a snap attachment member, a hook and eye attachment member, a nail and nail hole attachment member, a screw and nut attachment member, a split pin attachment member, a button, a zipper, a string and eye attachment member, a saw tooth attachment member, or any other suitable attachment member or combination of attachment members.

The holder body second surface 108 may have at least one holder locking element 116. As shown in FIGS. 1-3, the holder body second surface 108 may have first, second, and third holder locking elements 116a, 116b, 116c that may be laterally spaced about the holder unit longitudinal axis LO so that the holder unit 102 is rotationally symmetrical. Each holder locking element 116a, 116b, 116c includes a holder leg 118 (shown here as first, second and third holder legs 118a, 118b, 118c) and a holder foot 120 (shown here as first, second, and third holder feet 120a, 120b, 120c). Each of the first, the second, and the third holder legs 118a, 118b, 118c may longitudinally extend downward from the holder body second surface 108. The first, the second, and the third holder legs 118a, 118b, 118c may longitudinally extend downward from at least one of the holder body 104 and a corresponding holder body branch 110a, 110b, 110c. As shown in FIGS. 1-3, each of the first, the second, and the third holder legs 118a, 118b, 118c longitudinally extend downward from the holder body second surface 108 at a corresponding holder body branch 110a, 110b, 110c. In such case, the number of holder body legs and feet 118a, 118b, 118c, 120a, 120b, 120c may correspond to the number of holder body branches 110a, 110b, 110c. The first, the second, and the third holder legs 118a, 118b, 118c may each longitudinally extend between the holder body second surface 108 and a holder foot first surface 121 (shown here as first, second and third holder foot first surfaces 121a, 121b, 121c) of a corresponding holder foot 120a, 120b, 120c.

The odor control device 100 further includes a replaceable cartridge 422 that is infused with a deodorizer. The term “deodorizer” is defined herein as a substance or material that at least partially removes and/or masks odors. The cartridge 422 may have one of any number of alternate configurations, some of which will be discussed below. Regardless of the configuration, each configuration of the cartridge 422 has a cartridge first surface 424, a longitudinally spaced and oppositely facing cartridge second surface 426, and a cartridge side wall 428 that longitudinally extends between the cartridge first and second surfaces 424, 426. Each alternate configuration of the cartridge 422 may have at least one cartridge locking element 430 for at least partially selectively engaging the at least one holder locking element 116. The at least one holder locking element 116 is selectively engaged with the at least one cartridge locking element 424 to at least partially selectively attach the cartridge 422 to the holder unit 102 and at least partially selectively maintain the cartridge 422 in the holder unit 102.

The number of cartridge locking elements 430 may correspond to the number of holder locking elements 116. In such case, when the holder unit 102 has at least one holder locking element 116, the cartridge 422 may have at least one cartridge locking element 430. When the holder unit 102 has at least two holder locking elements 116, the cartridge 422 may have at least two cartridge locking elements 430. FIGS. 1-3 depict the holder unit 102 having the first, the second, and the third holder locking elements 116a, 116b, 116c. In such case, the cartridge 422 may have first, second, and third cartridge locking elements 430a, 430b, 430c.

The at least one cartridge locking element 430 may be at least one of integrally formed with and integrally formed in the cartridge 422. The phrase “integrally formed with” is defined herein as two or more elements being formed at the same time as one another so that they form one single piece. For example, the at least one cartridge locking element 430 and the cartridge 422 may be formed, and formed with one another, during a single molding process. The phrase “integrally formed in” is defined herein as a first element being made prior to a second element, and the second element being formed around and/or on the first element so that the second element at least partially encapsulates the first element. For example, the at least one cartridge locking element 430 may be formed prior to the cartridge 422. Once the at least one cartridge locking element 430 is formed, the cartridge 422 is overmolded around and/or over the at least one cartridge locking element 430 to at least partially encapsulate the at least one cartridge locking element 430.

Although each alternate configuration of the cartridge includes at least one cartridge locking element 430 for selectively attaching the cartridge 422 to the holder unit 102, some of the alternate configurations of the cartridge 422 may include alternate configurations for at least one of the cartridge 422 itself and the at least one cartridge locking element 430. Each of the alternate configurations of the cartridge 422 and the at least one cartridge locking element 430 may provide alternate sequences of operation for engaging the cartridge 422 to the holder unit 102.

FIGS. 4-6 depict an example alternate configuration for the cartridge 422, referred to as cartridge A 422′. The cartridge A 422′ may have at least one cartridge locking element 430, referred to as the at least one cartridge locking element 430′, for engaging the at least one holder locking element 116. The cartridge A 422′ is shown having at least two cartridge locking elements 430′ (shown here as first, second, and third holder locking elements 430a′, 430b′, 430c′). As shown in FIG. 4, the first, the second, and the third holder locking elements 430a′, 430b′, 430c′ may be laterally spaced about a cartridge longitudinal axis CA so that the cartridge A 422′ is rotationally symmetrical. The first, the second, and the third holder locking elements 430a′, 430b′, 430c′ are positioned on the cartridge A 422′ to selectively engage a corresponding holder locking element 116a, 116b, 116c. Each of the cartridge locking elements 430a′, 430b′, 430c′ may be an aperture 432 (shown here as first, second, and third apertures 432a, 432b, 432c) that longitudinally extends between the cartridge first and second surfaces 424, 426. Each of the apertures 432a, 432b, 432c is sized to receive a corresponding holder foot 120a, 120b, 120c and at least a portion of a corresponding holder leg 118a, 118b, 118c of the first, the second, and the third holder locking elements 116a, 116b, 116c.

The cartridge A 422′ may be formed from a deformable and elastomeric material. In such case, the cartridge A 422′ is selectively deflectable from a biased condition (FIG. 4). The deflection from the biased condition occurs responsive to at least one of an applied force and an elastic deformation of the cartridge A 422′. A user may apply a force to the cartridge A 422′ to deflect the cartridge A 422′ from the biased condition. Upon removal of the applied force, the cartridge A 422′ automatically moves or “springs” back to the biased condition from being deflected in response to the inherent properties of the elastomeric material. As shown in FIG. 4, the cartridge A 422′ may have at least one tab 434 that extends from at least one of the cartridge first surface 424, the cartridge second surface 426, and the cartridge side wall 428. The applied force may be selectively applicable to the at least one tab 434 to at least partially deflect the cartridge A 422′ from the biased condition.

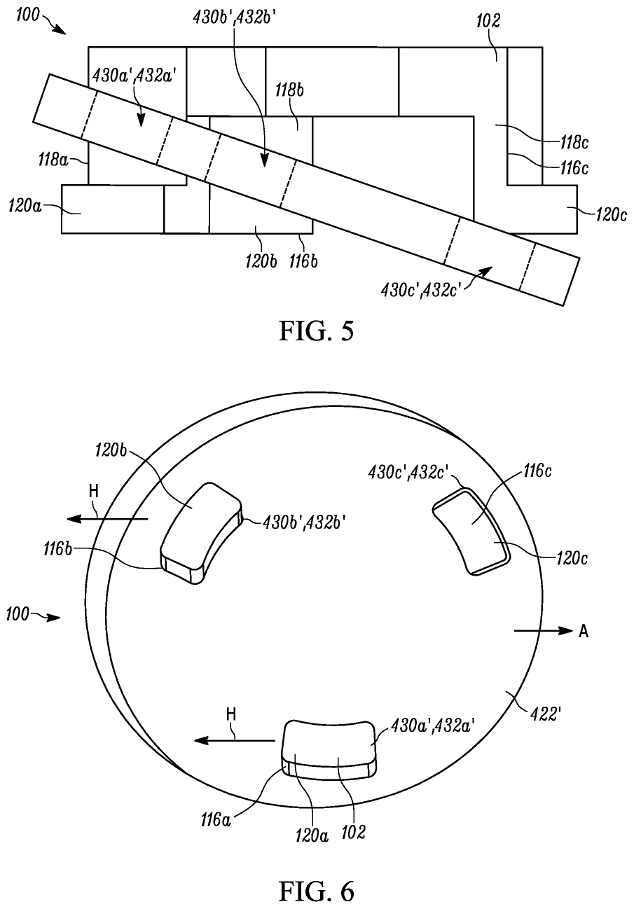

When the cartridge A 422′ is in the biased condition, a portion of the at least two cartridge locking elements 430a′, 430b′, 430c′ are selectively engageable to a portion of the holder locking elements 116a, 116b, 116c. As shown in FIG. 5, the first and the second cartridge locking elements 430a′, 430b′ are selectively engageable to the first and the second holder locking elements 116a, 116b when the cartridge A 422′ is in the biased condition. In particular, at least a portion of the first and second holder feet and legs 120a, 120b, 118a, 118b of the first and second holder locking elements 116a, 116b are selectively receivable in the first and second apertures 432a, 432b.

The cartridge A 422′ is selectively deflectable from the biased condition to selectively engage at least a portion of each of the at least two cartridge locking elements 430a′, 430b′, 430c′ to at least a portion of a corresponding one of the at least two holder locking elements 116a, 116b, 116c. In particular, when a portion of the cartridge locking elements 430a′, 430b′, 430c′ are engaged to a portion of the holder locking elements 116a, 116b, 116c, the portion of the holder locking elements 116a, 116b, 116c that is engaged to cartridge locking elements 430a′, 430b′, 430c′ provides a holder force in response to an applied force acting on the cartridge A 422′. The applied force and the holder force cooperatively cause the cartridge A 422′ to deflect from the biased condition to selectively engage at least a portion of the at least two cartridge locking elements 430a′, 430b′, 430c′ to at least a portion of the at least two holder locking elements 116a, 116b, 116c.

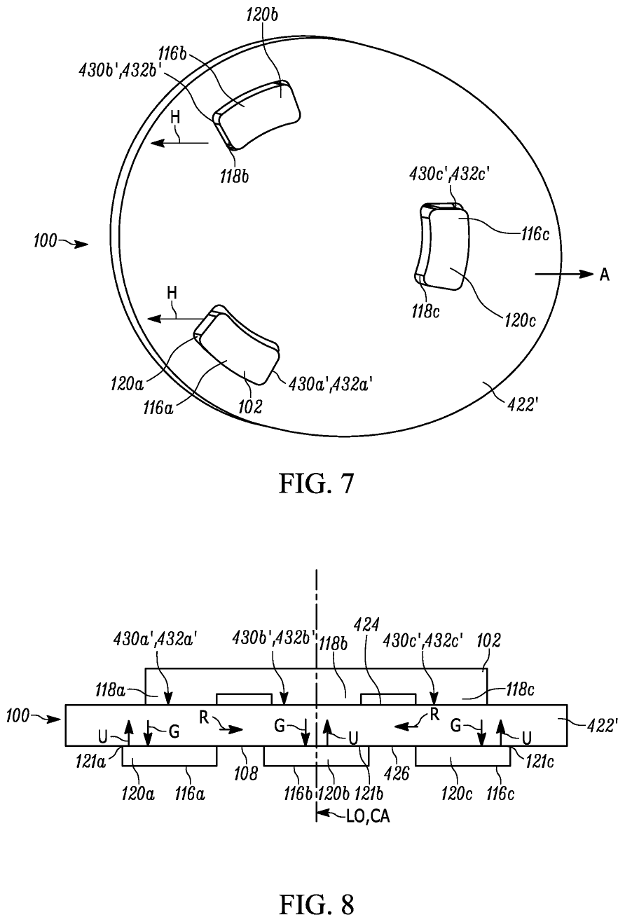

As shown in FIG. 6, the first and second cartridge locking elements 430a′, 430b′ are engaged to the first and the second holder locking elements 116a, 116b, such as by at least a portion of the first and second holder feet and legs 120a, 120b, 118a, 118b of the first and second holder locking elements 116a, 116b being received in the first and second apertures 432a, 432b. In response to an applied force (shown here as an arrow A in FIG. 6), the first and second holder locking elements 116a, 116b provide a holder force (shown here as arrows H in FIG. 6), such as through at least one of the first and second holder legs 118a, 118b and the first and second holder feet 120a, 120b of the first and second holder locking elements 116a, 116b. As shown in FIG. 7, the applied force and the holder force cooperatively cause the cartridge A 422′ to deflect from the biased condition to selectively engage the third cartridge locking element 430c′ to the third holder locking element 116c′, such as by receiving at least a portion of the third holder foot and leg 120c, 118c of the third holder locking element 116c in the third aperture 432c.

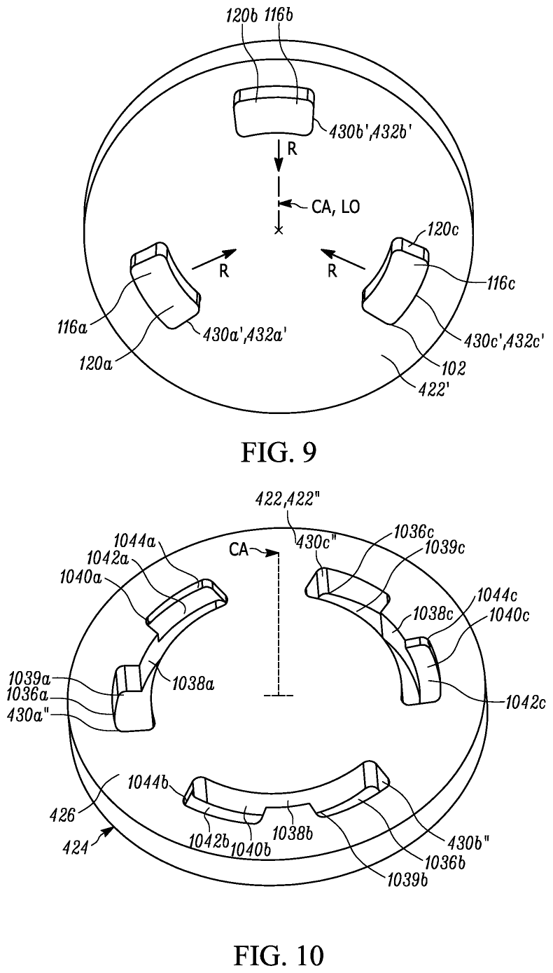

As shown in FIGS. 8-9, once the third holder locking element 116c′ is engaged to the third cartridge locking element 430c′, the applied force may be removed from the cartridge A 422′ so that the cartridge A 422′ may return to the biased condition with the first, the second, and the third holder locking elements 116a, 116b, 116c engaged to the first, the second, and the third cartridge locking elements 430a′, 430b′, 430c′. The engagement of the at least two cartridge locking elements 430a′, 430b′, 430c′ (e.g., the first, the second, and the third cartridge locking elements 430a′, 430b′, 430c′) to the at least two holder locking elements 116a, 116b, 116c (e.g., the first, the second, and the third holder locking elements 116a, 116b, 116c) at least partially maintains the cartridge A 422′ in the holder unit 102.

When the at least two holder locking elements 116a, 116b, 116c are engaged to the at least two cartridge locking elements 430a′, 430b′, 430c′, the innate elasticity of the elastomeric material of the cartridge A 422′ may provide a radially inward pressure from the at least two cartridge locking elements 430a′, 430b′, 430c′ to the at least two holder locking elements 116a, 116b, 116c. The term “radial” is used herein to indicate a direction substantially perpendicular to the “longitudinal” direction, and is shown via arrows R in FIGS. 8-9 emanating toward a central longitudinal axis CA, in the orientation of FIGS. 8-9. The radially inward pressure at least partially maintains the cartridge A 422′ in the holder unit 102. Further, when the at least two holder locking elements 116a, 116b, 116c are engaged to the at least two cartridge locking elements 430a′, 430b′, 430c′, at least a portion of each of the first, the second, and the third holder foot first surfaces 121a, 121b, 121c contacts at least a portion of the cartridge second surface 426. The cartridge second surface 426 exerts a longitudinally downward force (shown here as arrows G) on the portions of the first, the second, and the third holder foot first surfaces 121a, 121b, 121c that are contacting cartridge second surface 426 as a result of gravity acting on the cartridge A 422′. In response to the longitudinally downward force, the portions of the first, the second, and the third holder foot first surfaces 121a, 121b, 121c that are contacting the cartridge second surface 426 exerts a longitudinally upward force (shown here as arrows U) on the portions of the cartridge second surface 426 that are contacting the first, the second, and the third holder foot first surfaces 121a, 121b, 121c. The opposing forces cooperatively at least partially maintain the cartridge A 422′ in the holder unit 102.

Although the cartridge A 422′ has been described as deflecting as a result of the cooperation of the applied force and the holder force, the cartridge A 422′ may be deflected from a biased condition to engage at least one cartridge locking element 430′ to at least one holder locking element 116 through only an applied force and no holder force. For example, a holder unit 102 may have one holder locking element 116. In such case, the cartridge A 422′ may have one cartridge locking element 430′ that is not engageable to the holder locking element 116 when the cartridge A 422′ is in the biased condition. The cartridge A 422′ may be deflected from the biased condition by at least one applied force to engage the cartridge locking element 430′ to the holder locking element 116. Once the holder locking element 116 is engaged to the cartridge locking element 430′, the at least one applied force may be removed from the cartridge A 422′ so that the cartridge A 422′ may return to the biased condition with the holder locking element 116 engaged to the cartridge locking element 430′. With the holder locking element 116 engaged to the cartridge locking element 430′, the cartridge A 422′ is maintained in the holder unit 102 in a similar manner as described above.

Although the cartridge A 422′ has been described as being deflected in order to selectively engage the holder unit 102, it is contemplated that the at least one holder locking element 116a, 116b, 116c may be formed from a deformable and elastomeric material such that the at least one holder unit locking element 116a, 116b, 116c may be selectively deflectable in order to engage the at least one cartridge locking element 430a′, 430b′, 430c′ of the cartridge A 422′.

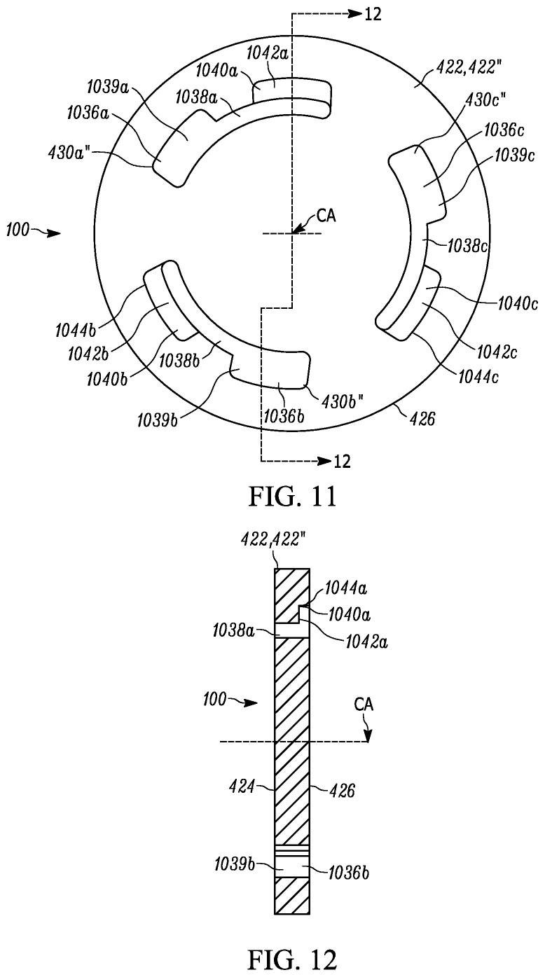

FIGS. 10-15 depict an example alternate configuration for the cartridge 422, referred to as cartridge B 422″. The cartridge B 422″ may have at least one cartridge locking element 430, referred to as the at least one cartridge locking element 430″, for engaging the at least one holder locking element 116. The cartridge B 422″ is shown having at least two cartridge locking elements 430″ (shown here as first, second, and third cartridge locking elements 430a″, 430b″, 430c″). As shown in FIGS. 10-12, the first, the second, and the third cartridge locking elements 430a″, 430b″, 430c″ may be laterally spaced about the cartridge longitudinal axis CA so that the cartridge B 422″ is rotationally symmetrical. The first, the second, and the third holder locking elements 430a″, 430b″, 430c″ are positioned on the cartridge B 422″ to selectively engage a corresponding holder locking element 116a, 116b, 116c.

Each of the first, the second, and the third cartridge locking elements 430a″, 430b″, 430c″ may have an insertion portion 1036 (shown here as first, second, and third insertion portions 1036a, 1036b, 1036c), a track 1038 (shown here as first, second, and third tracks 1038a, 1038b, 1038c), and a seat 1040 (shown here as first, second, and third seats 1040a, 1040b, 1040c). Each of the first, the second, and the third insertion portions 1036a, 1036b, 1036c may be an aperture 1039 (shown here as first, second, and third apertures 1039a, 1039b, 1039c) that at least partially longitudinally extends between the cartridge first and second surfaces 424, 426 and is sized to receive a corresponding holder locking element 116a, 116b, 116c. In particular, each of the first, the second, and the third apertures 1039a, 1039b, 1039c may be sized to receive a corresponding holder foot 120a, 120b, 120c and at least a portion of a corresponding holder leg 118a, 118b, 118c.

Each of the first, the second, and the third tracks 1038a, 1038b, 1038c at least partially longitudinally extends between the cartridge first and second surfaces 424, 426 and laterally extends between a corresponding insertion portion 1036a, 1036b, 1036c and a corresponding seat 1040a, 1040b, 1040c. The first, the second, and the third seats 1040a, 1040b, 1040c partially longitudinally extend between the cartridge first and second surfaces 424, 426. The first, the second, and the third seats 1040a, 1040b, 1040c each have a holder foot engagement surface 1042 (shown here as first, second, and third holder foot engagement surfaces 1042a, 1042b, 1042c) that is longitudinally spaced upward from the cartridge second surface 426, and has seat side walls (shown here as first, second, and third seat side walls 1044a, 1044b, 1044c) that longitudinally extend between a corresponding holder foot engagement surface 1042a, 1042b, 1042c and the cartridge second surface 426.

When the at least one holder locking element 116a, 116b, 116c is selectively received in the at least one cartridge locking element 430a″, 430b″, 430c″ of cartridge B 422″, the cartridge B 422″ is selectively laterally rotatable in a first direction to at least partially selectively engage the at least one cartridge locking element 430a″, 430b″, 430c″ with the at least one holder locking element 116a, 116b, 116c. The selective engagement of the at least one cartridge locking element 430a″, 430b″, 430c″ with the at least one holder locking element 116a, 116b, 116c at least partially maintains the cartridge B 422″ in the holder unit 102.

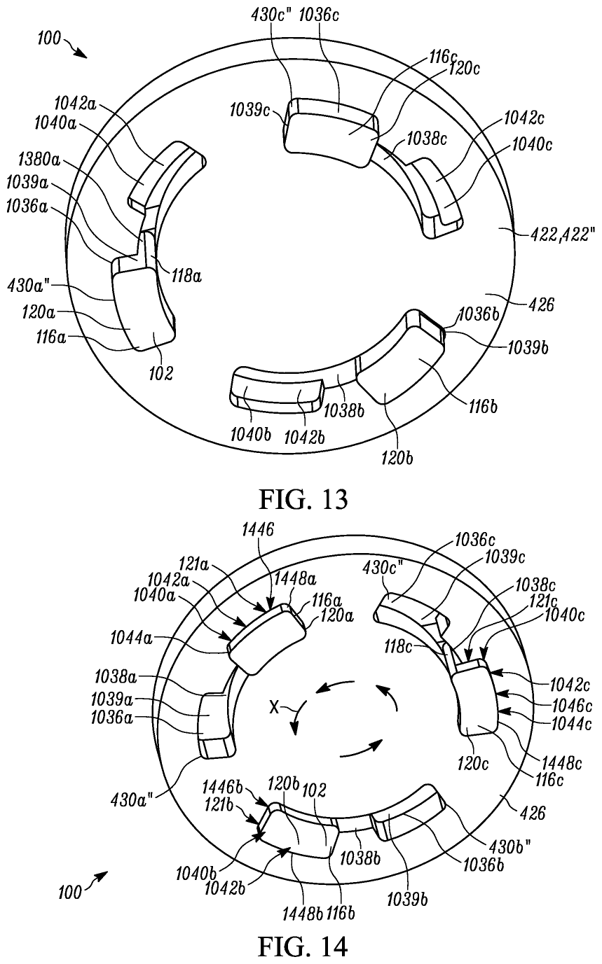

As shown in FIG. 13, each of the first, the second, and the third holder feet 120a, 120b, 120c and at least a portion of each of the first, the second, and the third holder legs 118a, 118b, 118c of the first, the second, and the third holder locking elements 116a, 116b, 116c are received in a corresponding insertion portion 1036a, 1036b, 1036c of a corresponding cartridge locking element 430a″, 430b″, 430c″. As shown in FIG. 14, when each of the first, the second, and the third holder feet 120a, 120b, 120c and at least a portion of each of the first, the second, and the third holder legs 118a, 118b, 118c are received in the first, the second, and the third insertion portions 1036a, 1036b, 1036c, the lateral rotation of the cartridge B 422″ in the first direction (shown here as arrows X) causes each of the first, the second, and the third tracks 1038a, 1038b, 1038c to move along at least one of a corresponding holder leg 118a, 118b, 118c and a corresponding holder foot 120a, 120b, 120c until each of the first, the second, and the third seats 1040a, 1040b, 1040c engages a corresponding holder foot 120a, 120b, 120c. When the first, the second, and the third seats 1040a, 1040b, 1040c engage the corresponding holder feet 120a, 120b, 120c, the first, the second, and the third holder foot engagement surfaces 1042a, 1042b, 1042c abut a corresponding cartridge engagement surface 1446 (shown here as first, second, and third cartridge engagement surfaces 1446a, 1446b, 1446c) on the holder foot first surface 121a, 121b, 121c of a corresponding holder foot 120a, 120b, 120c and the first, the second, and the third seat side walls 1044a, 1044b, 1044c are adjacent to at least a portion of holder foot side walls 1448 (shown here as first, second, and third holder foot side walls 1448a, 1448b, 1448c) of the corresponding holder feet 120a, 120b, 120c.

The engagement of the first, the second, and the third seats 1040a, 1040b, 1040c to the holder feet 120a, 120b, 120c at least partially maintains the cartridge B 422″ in the holder unit 102. In particular, as shown in FIG. 15, when the first, the second, and the third seats 1040a, 1040b, 1040c engage the holder feet 120a, 120b, 120c, the first, the second, and the third holder foot engagement surfaces 1042a, 1042b, 1042c of the first, the second, and the third seats 1040a, 1040b, 1040c exert a longitudinally downward force (shown here as arrows G) on the first, the second, and the third cartridge engagement surfaces 1446a, 1446b, 1446c of the holder feet 120a, 120b, 120c as a result of gravity acting on the cartridge B 422″. In response to the longitudinally downward force, the first, the second, and the third cartridge engagement surfaces 1446a, 1446b, 1446c exerts a longitudinally upward force (shown here as arrows U) on the first, the second, and the third holder foot engagement surfaces 1042a, 1042b, 1042c. The opposing forces cooperatively at least partially maintain the cartridge B 422″ in the holder unit 102. Further, when the first, the second, and the third seats 1040a, 1040b, 1040c engage the first, the second, and the third holder feet 120a, 120b, 120c, the first, the second, and the third seat side walls 1044a, 1044b, 1044c being adjacent to at least a portion of the first, the second, and the third holder foot side walls 1448a, 1448b, 1448c at least partially prevents the cartridge B 422″ from being rotated in a second direction (shown here as arrows Y) opposite to the first direction. When the cartridge B 422″ is at least partially prevented from being rotated in the second direction, the cartridge B 422″ is at least partially maintained in the holder unit 102.

Although the engagement of the cartridge 422 to the holder unit 102 has been described as being the result of one of a deflection (cartridge A 422′) and a lateral rotation (cartridge B 422″), any other engagement may be provided, such as, but not limited to, a magnetic engagement, a hook and loop engagement, a nail and nail hole engagement, a snap-fit engagement, an adhesive engagement, a screw and nut engagement, a split pin engagement, a button engagement, a press-fit engagement, any other selectively removable suitable engagement, or any combination thereof.

Although the cartridge 422 has been shown as having a cylindrical shape, it is contemplated that the cartridge 422 may have a cube shape, a cuboid shape, a triangular prism shape, or any other appropriate shape.

Although the holder unit 102 has been described as having features, such as the holder body branches 110a, 110b, 110c, when provided, and the holder locking elements 116a, 116b, 116c, that are laterally spaced about the holder body longitudinal axis LO so that the holder unit 102 is rotationally symmetrical, any of the features of the holder unit 102 may be laterally spaced about the holder body longitudinal axis LO in any desired manner. For example, the holder body branches 110a, 110b, 110c, when provided, and the holder locking elements 116a, 116b, 116c may be laterally spaced about the holder body longitudinal axis LO so that the holder body 102 is not rotationally symmetrical in order to be utilized with a corresponding device, product, and/or element.

Although the cartridges A and B 422′, 422″ have been described as having features, such as the cartridge locking elements 430a′, 430b′, 430c′, 430a″, 430b″, 430c″, that are laterally spaced about the cartridge longitudinal axis CA so that the cartridges A and B 422, 422″ are rotationally symmetrical, any of the features of the cartridges A and B 422′, 422″ may be laterally spaced about the cartridge longitudinal axis CA in any desired manner. For example, the cartridge locking elements 430a′, 430b′, 430c′, 430a″, 430b″, 430c″ may be laterally spaced about the cartridge longitudinal axis CA so that the cartridges A and B 422′, 422″ are not rotationally symmetrical in order to be utilized with a corresponding device, product, and/or element.

It is contemplated that at least one of the cartridge first surface 424, the cartridge second surface 426, and the cartridge side wall 428 of any of the alternate cartridge 422 configurations may include the at least one tab 434 to assist the user in manipulating the cartridge 422. It is also contemplated that at least one of the cartridge first surface 424, the cartridge second surface 426, and the cartridge side wall 428 of any of the alternate cartridge 422 configurations may be free from any protrusions, such as the at least one tab 434.

The holder unit 102 can be at least partially formed from silicone, polyethylene, polypropylene, polycarbonate, polyamides, stainless steel, titanium, rubber, any other suitable material, or any combination thereof. The cartridge 422 can be at least partially formed from cellulose, rubber, synthetic polymers, synthetic resins, thermoplastic resins, polyvinyl chloride, any other material capable of holding/containing a deodorizer, deodorizers, materials used to form conventional deodorizer blocks, or any combination thereof. Further, it is contemplated that the cartridge 422 may or may not be formed from a material that is capable of disintegrating over time.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

I claim:

1. An odor control device, comprising:
a holder unit having a holder body, the holder body having a holder body first surface and an oppositely facing holder body second surface, the holder body second surface having at least one holder locking element; and
a replaceable cartridge being infused with a deodorizer, the cartridge having at least one cartridge locking element for engaging the holder locking element, the cartridge locking element being at least one of integrally formed with and integrally formed in the cartridge;
wherein the at least one holder locking element is selectively engaged with the at least one cartridge locking element to at least partially maintain the cartridge in the holder unit.

2. The odor control device of claim 1, wherein the at least one holder locking element is selectively receivable in the at least one cartridge locking element, and when the at least one holder locking element is received in the at least one cartridge locking element, the cartridge is selectively laterally rotatable in a first direction to at least partially engage the at least one cartridge locking element with the at least one holder locking element.

3. The odor control device of claim 1, wherein the at least one holder locking elements is at least two holder locking elements, the at least one cartridge locking element is at least two holder locking elements, and the cartridge is at least partially formed from a deformable and elastomeric material, the cartridge being selectively deflectable from a biased condition to selectively engage at least a portion of each of the at least two cartridge locking elements to at least a portion of a corresponding one of the at least two holder locking elements, the engagement of the at least two cartridge locking elements to the at least two holder locking elements at least partially maintaining the cartridge in the holder unit.

4. The odor control device of claim 1, wherein the cartridge is at least partially formed from a deformable and elastomeric material, the cartridge being selectively deflectable from a biased condition to selectively engage at least a portion of the at least one cartridge locking element to at least a portion of the at least one holder locking element, the engagement of the at least one cartridge locking element to the at least one holder locking element at least partially maintaining the cartridge in the holder unit, and when the at least one holder locking element are received in the at least one cartridge locking element, the innate elasticity of the elastomeric material of the cartridge provides a radially inward pressure from the at least one cartridge locking element to the at least one holder locking element, the radially inward pressure at least partially maintaining the cartridge in the holder unit.

5. The odor control device of claim 1, wherein the holder body first surface has at least one attachment element for attaching the holder unit to a support surface.

6. The odor control device of claim 1, wherein the holder unit is provided as an integral part of a support surface such that the support surface comprises at least a portion of the holder body first surface.

7. An odor control device, comprising:
a holder unit having a holder body, the holder body having a holder body first surface and an oppositely facing holder body second surface, the holder body second surface having at least two holder locking elements; and
a replaceable cartridge being infused with a deodorizer and at least partially formed from a deformable and elastomeric material, the cartridge having at least two cartridge locking elements, the cartridge being selectively deflectable from a biased condition to selectively engage at least a portion of each of the at least two cartridge locking elements to at least a portion of a corresponding one of the at least two holder locking elements;
wherein the engagement of the at least two cartridge locking elements to the at least two holder locking elements at least partially maintains the cartridge in the holder unit.

8. The odor control unit of claim 7, wherein in the biased condition, a portion of the at least two cartridge locking elements are selectively engageable to a portion of the holder locking elements, and when a portion of the cartridge locking elements are engaged to a portion of the holder locking elements, the portion of the holder locking elements engaged to cartridge locking elements provide a holder force in response to an applied force acting on the cartridge, the applied force and the holder force cooperatively causing the cartridge to deflect from the biased condition to selectively engage at least a portion of the at least two cartridge locking elements to at least a portion of the at least two holder locking elements.

9. The odor control unit of claim 7, wherein each cartridge locking element of the at least two cartridge locking elements is an aperture that extends between a cartridge first surface and an oppositely facing cartridge second surface.

10. The odor control device of claim 9, wherein each holder locking element of the at least two holder locking elements includes a holder leg and a holder foot, the holder leg longitudinally extending downward from the holder body second surface, the holder leg longitudinally extending between the holder body second surface and a holder foot first surface, and each aperture is sized to receive a corresponding holder foot and at least a portion of a corresponding holder leg.

11. The odor control unit of claim 10, wherein when the at least two holder locking elements are received in the at least two cartridge locking elements, at least a portion of the holder foot first surface contacts cartridge second surface so that the cartridge second surface exerts a longitudinally downward force on the portion of the holder foot first surface contacting cartridge second surface, and the portion of the holder foot first surface contacting cartridge second surface exerts a longitudinally upward force on the portion of the cartridge second surface that is contacting the holder foot first surface, the opposing forces cooperatively at least partially maintaining the cartridge in the holder unit.

12. The odor control device of claim 7, wherein when the at least two holder locking elements are engaged to the at least two cartridge locking elements, the innate elasticity of the elastomeric material of the cartridge provides a radially inward pressure from the at least two cartridge locking elements to the at least two holder locking elements, the radially inward pressure at least partially maintaining the cartridge in the holder unit.

13. The odor control device of claim 7, wherein the cartridge has a cylindrical shape, the cartridge having a cartridge first surface and a longitudinally spaced and oppositely facing cartridge second surface, and the at least two cartridge locking elements are apertures that longitudinally extend between the cartridge first and second surfaces.

14. The odor control device of claim 13, wherein the cartridge has at least one tab extending from at least one of the cartridge first surface, the cartridge second surface, and a cartridge side wall that longitudinally extends between the cartridge first and second surfaces, and an applied force is selectively applicable to the tab to at least partially deflect the cartridge from the biased condition.

15. The odor control device of claim 7, wherein the holder body first surface has at least one attachment element for attaching the holder unit to a support surface.

16. The odor control device of claim 15, wherein the attachment element is an adhesive, the adhesive being covered by a selectively removable cover when the odor control device is in a stored condition.

17. The odor control device of claim 7, wherein the holder unit is provided as an integral part of a support surface such that the support surface comprises at least a portion of the holder body first surface.

18. An odor control device, comprising:
a holder unit having a holder body, the holder body having a holder body first surface and an oppositely facing holder body second surface, the holder body second surface having at least one holder locking element; and
a replaceable cartridge being infused with a deodorizer, the cartridge having at least one cartridge locking element for receiving the holder locking element;
wherein when the at least one holder locking element is selectively received in the at least one cartridge locking element, the cartridge is selectively laterally rotatable in a first direction to at least partially engage the at least one cartridge locking element with the at least one holder locking element, the engagement of the at least one cartridge locking element with the at least one holder locking element at least partially maintaining the cartridge in the holder unit.

19. The odor control device of claim 18, wherein the at least one cartridge locking element includes:
an insertion portion at least partially longitudinally extending between a cartridge first surface and a cartridge second surface, the cartridge second surface being oppositely facing from the cartridge first surface, the insertion portion being sized to receive the at least one holder locking element; and
a track at least partially longitudinally extending between the cartridge first and second surfaces, the track laterally extending between the insertion portion and a seat so that when at least a portion of the at least one holder locking element is received in the insertion portion, the lateral rotation of the cartridge in the first direction causes the track to move along at least a portion of the at least one holder locking element until the seat engages at least a portion of the at least one holder locking element, the engagement of the seat to at least a portion of the at least one holder locking element at least partially maintaining the cartridge in the holder unit.

20. The odor control device of claim 18, wherein the at least one holder locking element includes a holder leg and a holder foot, the holder leg longitudinally extending downward from the holder body second surface, the holder leg longitudinally extending between the holder body second surface and a holder foot first surface.

21. The odor control device of claim 20, wherein the at least one cartridge locking element includes:
an insertion portion at least partially longitudinally extending between a cartridge first surface and a cartridge second surface, the cartridge second surface being oppositely facing from the cartridge first surface, the insertion portion being sized to receive the holder foot and at least a portion of the holder leg; and
a track at least partially longitudinally extending between the cartridge first and second surfaces, the track laterally extending between the insertion portion and a seat so that when the holder foot and at least a portion of the holder leg are received in the insertion portion, the lateral rotation of the cartridge in the first direction causes the track to move along at least one of the holder leg and the holder foot until the seat engages the holder foot, the engagement of the seat to the holder foot at least partially maintaining the cartridge in the holder unit.

22. The odor control device of claim 21, wherein the seat partially longitudinally extends between the cartridge first and second surfaces, the seat having a holder foot engagement surface that is longitudinally spaced upward from the cartridge second surface, the holder foot engagement surface abutting a cartridge engagement surface on the first surface of the holder foot when the seat engages the holder foot, the seat having seat side walls that longitudinally extend between the holder foot engagement surface and the cartridge second surface, the seat side walls being adjacent to at least a portion of holder foot side walls when the seat engages the holder foot to at least partially prevent the cartridge from being rotated in a second direction and at least partially maintain the cartridge in the holder unit, the second direction being opposite to the first direction.

23. The odor control device of claim 22, wherein when the seat engages the holder foot, the holder foot engagement surface of the seat exerts a longitudinally downward force on the cartridge engagement surface of the holder foot, and the cartridge engagement surface of the holder foot exerts a longitudinally upward force on the holder foot engagement surface of the seat, the opposing forces cooperatively at least partially maintaining the cartridge in the holder unit.

24. The odor control device of claim 18, wherein the number of holder locking elements corresponds to the number of cartridge locking elements, each cartridge locking element being positioned on the cartridge to receive a corresponding holder locking element.

25. The odor control device of claim 18, wherein the at least one holder unit locking member comprises first, second, and third holder locking elements that are laterally spaced about a holder unit longitudinal axis so that the holder unit is rotationally symmetrical, the at least one cartridge locking element comprising first, second, and third cartridge locking elements that are laterally spaced about a cartridge longitudinal axis so that the cartridge is rotationally symmetrical, each cartridge locking element being positioned on the cartridge to receive a corresponding holder locking element.

26. The odor control device of claim 18, wherein the holder body first surface has at least one attachment element for attaching the holder unit to a support surface.

27. The odor control device of claim 26, wherein the attachment member is an adhesive, the adhesive being covered by a selectively removable cover when the odor control device is in a stored condition.

28. The odor control device of claim 18, wherein the holder unit is provided as an integral part of a support surface such that the support surface comprises at least a portion of the holder body first surface.

29. The odor control device of claim 18, wherein the cartridge has a cylindrical shape, the cartridge having a cartridge first surface and a longitudinally spaced and oppositely facing cartridge second surface, and the at least one cartridge locking element is an aperture that longitudinally extends between the cartridge first and second surfaces.

30. The odor control device of claim 29, wherein the cartridge first and second surfaces are free from protrusions.
